(19) United States
(12) Patent Application Publication
(10) Pub. No.: US 2018/0214481 A1
(43) Pub. Date: Aug. 2, 2018

(54) READY TO USE SURFACE DISINFECTANT

(71) Applicant: TUCKER D. MAURER, INDIANAPOLIS, IN (US)

(72) Inventor: TUCKER D. MAURER, INDIANAPOLIS, IN (US)

(73) Assignee: SPECTRA SHIELD TECHNOLOGIES, LLC, SCOTTSDALE, AZ (US)

(21) Appl. No.: 15/419,725

(22) Filed: Jan. 30, 2017

(51) Int. Cl. A61K 33/40; A61K 47/18; A61K 47/02; A61K 47/24

(52) U.S. Cl. CPC A61K 33/40; A61K 47/24; A61K 47/02; A61K 47/186

(57) ABSTRACT

This invention relates generally to improved chemical formulations and methods of use that provide for the disinfection of microorganisms and biological contaminants as well as the chemical neutralization of toxic materials (toxants) present on selected objects and surfaces. The invention relates more specifically to accelerated hydrogen peroxide based formulations having a secondary biocidal agent present and which demonstrate both desirous enhanced long term storage stability as well as efficacy of action at time of use without requiring the mixing or combination of the separated components immediate prior to use.

### BACKGROUND

### Field of the Invention

This invention relates generally to improved chemical formulations and methods of use that provide for the disinfection of microorganisms and biological contaminants as well as the chemical neutralization of toxic materials (toxants) present on selected objects and surfaces. The invention relates more specifically to accelerated hydrogen peroxide based formulations having a secondary biocidal agent present and which demonstrate extended long term storage stability and correspondingly enhanced efficacy of action at time of use without requiring the mixing or combination of the separated components immediate prior to use.

### Description of the Related Art

Numerous types of chemical formulations are known in the art that provide effective neutralization of chemical and biological toxants on objects and surfaces. Illustrative examples of comparative compositions are disclosed in U.S. Pat. Nos. 7,390,432, 7,271,137, 7,125,497, 6,723,890 and 6,566,574, which are each included herein in their entirety by reference. The traditional approach of using hydrogen peroxide based treatment compositions is to either stabilize the peroxide active by means of pH and buffering control, or to separate the peroxide active from other materials by means of separate treatment solution parts utilizing separated storage containers or containment devices that store two or more solution parts independently from one another until mixed or combined at the point of application, the latter for example being achievable by means of dual packaging systems which combine the separated liquid components only when applied or sprayed at time of use using a dual trigger sprayer with two dip tubes and a mixing valve, an example being the product Decon 7, a dual compartment, dual dip-tube spray-type packaged disinfectant combination product sold by Decon Seven Systems (10611 N. Hayden Road, Suite D-106, Scottsdale, Ariz. 85260 USA). These approaches, while allowing greater flexibility in formulation choices, suffer from the added packaging expense, requirement of mixing prior to use, and are subject to mechanical issues including disproportionate mixing ratios, changing ratios with fluid height and volume, as well as mechanical fatigue and failure of the mixing mechanisms. Further, undesirable cross-mixing of components can occur, otherwise requiring yet additional one-way valves to be included to prevent one solution part from contaminating the other during mixing and dispensing operations.

Accordingly, there is a high demand and need for comparable disinfecting and decontaminating systems that are ready to use and do not employ compositions that require dual storage or separated storage conditions prior to use. However, achieving a single solution approach in the presence of a highly reactive biocidal material such as hydrogen peroxide presents many challenges both in regard to stabilizing the compositions as well as in formulating ready to use compositions that can deliver efficacy of action at time of use, and particularly after long storage times before use.

### SUMMARY OF THE INVENTION

The instant invention addresses the need for a general formulation that neutralizes the adverse effects of either or both chemical and biological toxants, where a toxant is defined as any chemical or biological compound, constituent, species, or agent that through its chemical or biological action on life processes can, if left untreated, cause death, temporary incapacitation, or permanent harm to humans or animals. This includes all such chemicals or biological agents, regardless of their origin or of their method of production, and regardless of whether they are produced in facilities, in munitions or elsewhere. Neutralization is defined as the mitigation, detoxification, decontamination, or otherwise destruction of toxants to the extent that the toxants no longer cause acute adverse effects to humans or animals. The formulation and described variations of the instant invention can neutralize, and does not itself contain or produce, infection, significant adverse health effects, or even fatality in animals.

An important subset of chemical and biological compounds that the instant invention addresses is that of chemical warfare (“OW”) and biological warfare (“SW”) agents. However, the instant invention also addresses toxants that can cause potential adverse health effects to animals, including humans, where such adverse health effects include infections, acute and chronic health effects, and fatalities. Such toxants can be found in an agricultural facility, animal or dairy farm, or food products processing or packaging facility. Additionally, the instant invention addresses the need for such a formulation that is itself non-toxic and non-corrosive, and that can be delivered by a variety of means and in different phases.

One object of the instant invention is a formulation for use in the neutralization of at least one microorganism or toxant present on a surface, said formulation comprising: (a) a biocidal cationic surfactant; (b) a source of hydrogen peroxide, wherein said source of hydrogen peroxide is selected from the group consisting of hydrogen peroxide, peracetic acid, sodium perborate, sodium peroxypyrophosphate, sodium peroxysilicate, and sodium percarbonate; and (c) at least one oxidant-stable stabilizer; wherein said stabilizer is present at a level sufficient to stabilize said formulation against significant loss of said reactive compound for up to one year storage under ambient room temperature conditions; and wherein said stabilizer is present at or below a selected ratio with respect to said source of hydrogen peroxide enabling for accelerated oxidant activity when said formulation is applied to said surface at time of use; and wherein said ratio of hydrogen peroxide to stabilizer is less than or equal to 125:1.

A second object of the instant invention is a formulation according to any one embodiment disclosed herein having a ratio of hydrogen peroxide to stabilizer is equal to or between 90:1 and 125:1.

A further object of the instant invention is a formulation according to any one embodiment disclosed herein having a ratio of hydrogen peroxide to stabilizer is equal to or between 100:1 and 125:1

Another object of the instant invention is a formulation according to any one embodiment disclosed herein wherein said biocidal cationic surfactant is selected from the group consisting of dialkyl dimethyl ammonium salts, dialkyl diethyl ammonium salts, alkyl dialkylbenzyl ammonium salts, alkyl pyridinium ammonium salts, polyhexamethylene biguanide hydrochloride salts, polyhexamethylene guanidine hydrochloride salts, dimethyldidecyl ammonium chloride salts, benzalkonium chloride, benzethonium chloride, chlorhexidine digluconate, poly (dimethyl butenyl ammonium chloride salts, alpha, omega-bis (triethanol-ammonium chloride salts, poly (oxyethylene) (dimethylimino) ethylene (dimethylimino) ethylene dichloride, dequalinium chloride, polyquaternium 2, hexetidine, cetyl pyridinium chloride, tetrakis (hydroxy methyl) phosphonium sulfate, ethanediyl-α, w-bis (dodecyldimethyl) ammonium halides, quaternary ammonium dendrimeric biocides, and combinations thereof; wherein said alkyl substituents have saturated carbon chain lengths of 1 to 18, and combinations thereof.

Yet another object of the instant invention is a formulation according to any one embodiment disclosed herein wherein said biocidal cationic surfactant is selected from an N-alkyl dimethylbenzyl ammonium salt; wherein said N-alkyl substituent is a saturated alkyl substituent having a chain length equal to or between 12 and 18 alkane units; and wherein said salt is a counterion or adduct selected from a halide, hydroxide, sulfate, bicarbonate, carbonate or hydrogen chloride; and wherein said halide is either a chloride or bromide counterion.

A further object of the instant invention is a formulation according to any one embodiment disclosed herein wherein the biocidal cationic surfactant is selected from the group consisting of mixed alkyl substituent chain lengths, wherein said mixed alkyl substituent chain lengths include dodecyl, tetradecyl, and hexadecyl substituents.

Another object of the instant invention is a formulation according to any one embodiment disclosed herein wherein said N-alkyl dimethylbenzyl ammonium salt has a halide counterion; and wherein said N-alkyl substituent is a mixture of 40% dodecyl (C12), 50% tetradecyl (C14) and 10% hexadecyl (C16) substituents; and wherein said halide is either a chloride or bromide counterion.

Yet another object of the instant invention is a formulation according to any one embodiment disclosed wherein said biocidal cationic surfactant is present at a level equal to or between 0.05 weight % and 0.20 weight %.

One additional object of the instant invention is a formulation according to any one embodiment disclosed wherein said oxidant-stable stabilizer is selected from the group consisting of organic phosphonates, alkanehydroxyphosphonates, carboxylates, and mixtures thereof.

Another object of the instant invention is a formulation according to any one embodiment disclosed herein wherein said oxidant-stable stabilizer is selected from 1-hydroxethane-1,1-diphosphonic acid, amino-trismethylenephosphonic acid, diethylenetriamine penta(methylene phosphonic acid), diethylenetriamine penta(methylene phosphonic acid, hepta-sodium diethylenetriamine penta(methylene phosphonate, ethylenediaminetetraacetic acid, N-hydroxyethylene-diaminetetraacetic acid, nitrilotriacetic acid, N-hydroxyethyl-aminodiacetic acid, diethylene-triaminepentaacetic acid, methylglycinediacetic acid, alanine-N,N-diacetic acid, salts thereof, and combinations thereof.

Yet another object of the instant invention is a formulation according to any one embodiment disclosed herein wherein said oxidant-stable stabilizer is 1-hydroxethane-1,1-diphosphonic acid and salts thereof.

A further other object of the instant invention is a formulation according to any one embodiment disclosed herein wherein said oxidant-stable stabilizer is selected from a 1-hydroxethane-1,1-diphosphonic acid and salts thereof; wherein said biocidal cationic surfactant is an N-alkyl dimethylbenzyl ammonium salt having a halide counterion; wherein said N-alkyl substituent is a mixture of 40% dodecyl (C12), 50% tetradecyl (C14) and 10% hexadecyl (C16) substituents; and wherein said halide is either a chloride or bromide counterion.

Yet a further object of the instant invention is a formulation according to any one embodiment disclosed herein for use in neutralization of at least one microorganism or toxant present on a surface, said formulation consisting of: (a) a biocidal cationic surfactant being an N-alkyl dimethylbenzyl ammonium salt having a halide counterion; wherein said N-alkyl substituent is a mixture of 40% dodecyl (C12), 50% tetradecyl (C14) and 10% hexadecyl (C16) substituents; wherein said halide is either a chloride or bromide counterion; (b) a source of hydrogen peroxide, wherein said source of hydrogen peroxide is selected from the group consisting of hydrogen peroxide, peracetic acid, sodium perborate, sodium peroxypyrophosphate, sodium peroxysilicate, and sodium percarbonate; and (c) at least one oxidant-stable stabilizer being 1-hydroxethane-1,1-diphosphonic acid and salts thereof; wherein the ratio of said hydrogen peroxide to said stabilizer is equal to or between 95:1 and 125:1.

Another object of the instant invention is a formulation according to any one embodiment disclosed herein for use in neutralization of at least one microorganism or toxant present on a surface, where the formulation consists essentially of: (a) N-alkyl dimethylbenzyl ammonium chloride wherein said N-alkyl substituent is a mixture of 40% dodecyl (C12), 50% tetradecyl (C14) and 10% hexadecyl (C16) present at a level equal to or between 0.10 weight % and 0.13 weight %; (b) hydrogen peroxide present at a level equal to or between 4.5 weight % and 5.75 weight %; and (c) an oxidant-stable stabilizer selected from 1-hydroxethane-1,1-diphosphonic acid and salts thereof; wherein the ratio of said hydrogen peroxide to said oxidant-stable stabilizer is equal to or between 90:1 and 125:1.

A further object of the instant invention is a formulation according to any one embodiment disclosed herein enabling a method of treating a surface to neutralize at least one bacterial or chemical toxant, comprising the steps of (a) applying a formulation to said surface in the form of a thin aqueous film; wherein said formulation comprises: (i) a biocidal cationic surfactant; (ii) a source of hydrogen peroxide; wherein said source of hydrogen peroxide is selected from the group consisting of hydrogen peroxide, peracetic acid, sodium perborate, sodium peroxypyrophosphate, sodium peroxysilicate, and sodium percarbonate; (iii) at least one oxidant-stable stabilizer; wherein the ratio of said hydrogen peroxide to said oxidant-stable stabilizer is equal to or between 90:1 and 125:1; followed by the step (b) of allowing said thin aqueous film to remain in contact with said surface for a proscribed period of time of between 30 seconds and 10 minutes as necessary to neutralize said bacterial or chemical toxant; and a final step of (c) either removing said thin aqueous film from said surface or allowing said thin aqueous film to evaporate to dryness.

Yet a further object of the instant invention is a method of using a formulation to treat a surface wherein the biocidal cationic surfactant is selected from an N-alkyl dimethylbenzyl ammonium salt; wherein said N-alkyl substituent is a saturated alkyl substituent having a chain length equal to or between 12 and 18 alkane units; and wherein said salt is a counterion or adduct selected from a halide, hydroxide, sulfate, bicarbonate, carbonate or hydrogen chloride; wherein said halide is either a chloride or bromide counterion; and wherein said oxidant-stable stabilizer is selected from 1-hydroxethane-1,1-diphosphonic acid, amino-trismethylenephosphonic acid, diethylenetriamine penta(methylene phosphonic acid), diethylenetriamine penta(methylene phosphonic acid, hepta-sodium diethylenetriamine penta(methylene phosphonate, ethylenediaminetetraacetic acid, N-hydroxyethylene-diaminetetraacetic acid, nitrilotriacetic acid, N-hydroxyethyl-aminodiacetic acid, diethylene-triaminepentaacetic acid, methylglycinediacetic acid, alanine-N,N-diacetic acid, salts thereof, and combinations thereof.

Another object of the instant invention is a formulation according to any one embodiment disclosed herein which comprises (a) N-alkyl dimethylbenzyl ammonium chloride wherein said N-alkyl substituent is a mixture of 40% dodecyl (C12), 50% tetradecyl (C14) and 10% hexadecyl (C16) present at a level equal to or between 0.10 weight % and 0.13 weight %; (b) hydrogen peroxide present at a level equal to or between 4.5 weight % and 5.75 weight %; and (c) an oxidant-stable stabilizer selected from 1-hydroxethane-1,1-diphosphonic acid and salts thereof; wherein the ratio of said hydrogen peroxide to said oxidant-stable stabilizer is equal to or between 90:1 and 125:1.

A final object of the instant invention is a formulation according to any one embodiment disclosed herein whose method of use further includes instructions for applying and leaving said formulation in contact with a surface for at least two minutes in order to effect a 4-log reduction with respect to a colony formation unit of at least one microorganism selected from the genus Escherichia coli, Staphylococcus aureus, Pseudomonas aeruginosa, Salmonella enterica, and subgenera thereof, and subspecies thereof.

Further features and advantages of the instant invention will become apparent to those of ordinary skill in the art in view of the detailed description of preferred embodiments below, when considered together with the attached drawings and claims.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 is a plot of hydrogen peroxide stability of various compositions as a function of storage time and storage condition.

These and other objects and advantages of the instant invention will become more fully apparent from the following description taken in conjunction with the accompanying drawings. Reference will now be made to the drawings wherein like numerals refer to like parts throughout.

### DETAILED DESCRIPTION

Before describing the instant invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. Although a number of methods and materials similar or equivalent to those described herein can be used in the practice of the instant invention, the preferred materials and methods are described herein.

In the application, effective amounts are generally those amounts listed as the ranges or levels of ingredients in the descriptions, which follow herein. Unless otherwise stated, amounts listed in percentage (“%'s”) are in weight percent (weight % or wt. %) based on 100% activity and are expressed as wt. %/wt. % rather than by volume % of the total composition. In some instances, ingredients are expressed in terms of part per million (ppm) rather than weight %, noting that 1.0 wt. % is equivalent to 10,000 ppm for ease of comparison.

As used herein, the terms “chelant”, “sequestrant” and “builder” are intended to mean and to include any materials that can chelate or sequester metals or cationic metal counterions in aqueous solution, and which are generally termed and described as “stabilizer” with respect to their general property to act as stabilizers of active oxygen solutions, including hydrogen peroxide solutions.

The terms “oxidant”, “oxidative species”, “oxidation potential”, “redox potential”, as used herein, are intended to mean and to include the property and ability of a molecular species to either receive or transfer electrons (an oxidation and reduction or “redox” reaction) to a second molecular species, resulting in a change of oxidation state, or oxidation potential, or resulting in the chemical alteration or decomposition of the second molecular species reacting with and oxidant, oxidative species or chemical element or compound having an oxidation potential. Note that radical species are denoted with a ‘dot’ to represent a free electrons, regardless of the other change of the molecule or radical.

The term “disinfectant composition”, as used herein, is intended to include compositions having at least one property selected from antimicrobial, antifungal, antiviral, bactericidal, germicidal, virucidal, sporicidal and combinations therefore, with respect to antigens, archeons, bacteria, molds, microorganisms, fungus, prions, spores, viruses, and the like, including their by-products.

The term “formulation” is defined herein as the formulated product or solution (e.g., aqueous solution) that is ready to use as is, and can be directly applied to a surface or object for the purpose of neutralization of a toxant or biological contaminant or for the control of microorganisms.

The term “cleaning composition”, as used herein, is meant to mean and include a cleaning formulation having at least one surfactant.

The term “surfactant”, as used herein, is meant to mean and include a substance or compound that reduces surface tension when dissolved in water or water solutions, or that reduces interfacial tension between two liquids, or between a liquid and a solid. The term “surfactant” thus includes anionic, cationic, nonionic, and/or amphoteric agents, the latter being materials whose net charge varies as a function of the solution pH in which they are present.

The term “toxant”, as used herein, is meant to mean and include biological organisms, chemicals, and chemical and biological agents used as weapons.

The term “equivalent” (“eq”) and “milliequivalent” (“meq”), as used herein, is meant to define the number of molar equivalents with respect to a titratable quantity of a material to be standardized and that is expressed either in terms of normality (N) or molarity (M) units, which are typically dimensionless quantities.

### Examples

Embodiments of the inventive compositions disclosed herein employ at least two materials having some degree of activity against a biological organism or a toxant, having a first source of active oxygen or hydrogen peroxide, and a second source of a cationic alkyl ammonium quaternary biocide. In addition, embodiments of the inventive compositions disclosed herein include at least one stabilizing material selected from the group consisting of chelants, sequestrants, builders and the like. The embodiments of the instant invention are aqueous based solutions with water being the continuous solvent, and all ingredients present in a single, ready to use composition for ease of storage and application.

### Materials—Quaternary Amine

Biocidal quaternary amines suitable for use in compositions of the instant invention are readily available from a variety of sources, and include the class of dialkyl dimethyl ammonium salts, dialkyl diethyl ammonium salts, alkyl dialkylbenzyl ammonium salts, alkyl pyridinium ammonium salts, and mixtures thereof. Typically, these materials are commercially available as the halide salts, such as for example, chloride, bromide. Some additional suitable materials for use herein are also commercially available with other anionic cations, including hydroxide, acetate, and as hydrochloride adducts. Also suitable are polyhexamethylene biguanide hydrochloride salts, polyhexamethylene guanidine hydrochloride salts, dimethyldidecyl ammonium chloride salts, benzalkonium chloride, benzethonium chloride, chlorhexidine digluconate, poly (dimethyl butenyl ammonium chloride salts, alpha, omega-bis (triethanol-ammonium chloride salts, poly (oxyethylene) (dimethylimino) ethylene (dimethylimino) ethylene dichloride, dequalinium chloride, polyquaternium 2, hexetidine, cetyl pyridinium chloride, tetrakis (hydroxy methyl) phosphonium sulfate, gemini quats, e.g., ethanediyl-α, w-bis (dodecyldimethyl) ammonium halides, quaternary ammonium dendrimeric biocides (disclosed in U.S. Pat. No. 6,440,405, which is hereby incorporated in its entirety by reference) and mixtures thereof.

Also suitable for use in embodiments of the instant invention are more traditional mixed alkyl chain length cationic ammonium biocides, including for example, but not limited to Marquat MC1412-50%, an alkyl dimethylbenzyl ammonium chloride [CAS 6824-85-1] which is quaternary mixture of three major alkyl chain lengths, available from Pilot Chemical Company (2744 East Kemper Road, Cincinnati, Ohio 4524 USA), which has the following relative percentage distribution of alkyl chain lengths: 40% C12; 50% C14 and 10% C16. This material also has a small amount of ethanol [CAS 64-17-5] present reported at around 5.0 wt. %. Also suitable for use herein is Maquat® 2420-50%, which is a 50% active ADBAC, 40% n-Alkyl (40% C12, 50% C14, 10% C16) dimethylbenzyl ammonium chloride. Both of these materials have the advantage of prior registration with the Environmental Protection Agency under EPA Reg.#10324-6.

The specific biocides described are illustrative of this invention, but do not present a complete inventory of all the possible combinations possible. Anyone skilled in the art of chemistry and biology can conceptualize other modifications suitable for use herein.

The level of the quaternary amine biocide used in embodiments of the instant invention typically ranges, on an active basis, from between 0.05 wt. % to 0.20 wt. %, or alternatively from between 0.05 wt. % to 0.15 wt. %, or alternatively from between 0.10 wt. % to 0.125 wt. %, depending on the ultimate intended use.

One additional advantage of employing cationic ammonium quaternary biocides in embodiments of the instant invention is that these materials are surface active, thus also having surfactant-like properties that enable treatment solutions containing them to effectively wet and spread the treatment compositions uniformly across surfaces contacted by the aqueous compositions, regardless of the surfaces being non-polar or non-wettable by nature, such as for example, plastic or polymeric surfaces or objects not readily wetted with water alone. This surfactant property of the quaternary amine biocides is advantageous in ensuring that the inventive compositions come into uniform contact and spread across surfaces to be treated, so that no toxants or microbes are left unexposed to the inventive composition during treatment of a contaminated surface.

### Source of Hydrogen Peroxide

A source of active oxygen capable of producing hydrogen peroxide is preferred for use in embodiments of the instant invention. Hydrogen peroxide in the form of an aqueous solution is readily obtainable from a number of sources, but in some embodiments of the instant invention highly purified and stabilized material is preferred, antimicrobial and food grade materials having low levels of trace metals and other contaminants providing for improved storage longevity and compatibility with other materials present in the inventive compositions. In other embodiments, other sources of hydrogen peroxide are perfectly suitable, and the examples provided herein are for illustration purposes only and not a limitation on the materials of choice.

Suitable for use in embodiments of this invention are peroxide compounds that generate hydrogen peroxide in situ, such as for example, but not limited to, urea hydrogen peroxide; sodium perborate; sodium percarbonate; sodium carbonate perhydrate; sodium peroxypyrophosphate; sodium peroxysilicate hydrogen; peroxide adducts of pyrophosphates; citrates; sodium sulfate; urea; and sodium silicate; an activated peroxide compound (e.g., hydrogen peroxide+bicarbonate); peracetic acid; oximates (e.g., butane-2,3-dione, monooximate ion, and benzohydroxamate); alkoxides (e.g., methoxide and ethoxide); aryloxides (e.g., aryl substituted benzenesulfonates); aldehydes (e.g., glutaraldehyde) and peroxymonosulfate.

For example, in one embodiment of the instant invention, a solution of aqueous hydrogen peroxide [CAS 7722-84-1] is suitable, obtainable at a concentration of approximately 50 wt. % activity, designated as B-Cap™ 50 Antimicrobial Grade Hydrogen Peroxide, available from PeroxyChem (One Commerce Square, 2005 Market Street—Suite 3200, Philadelphia, Pa. 19103 USA), or under the same brand from FMC Corporation (1301 Ogletown Rd., Newark, Del. 19711 USA). In other embodiments of the instant invention, sources of aqueous hydrogen peroxide include for example, but are not limited to, selected grades available from Arkema (Arkema Inc., 900 First Avenue, King of Prussia, Pa. 19406 USA), INTEROX® SG-50 Grade Hydrogen Peroxide for pharmaceutical aseptic packaging applications available from Solvay (Rue de Ransbeek 310, 1120 Bruxelles, Belgium), Durox® 50% Hydrogen Peroxide, food grade, available from PeroxyChem, and Hydrox® hydrogen peroxide products available from Evonik Industries AG (Rellinghauser Straße 1-11, 45128 Essen, Germany).

Other sources of hydrogen peroxide include materials that release this active compound when dissolved or mixed into an aqueous solution. Representative source materials included herein above are, without being bound by theory, effective oxidants because they can produce a variety of negatively-charged nucleophiles, e.g., hydroxyl ions (OH⁻) and hydroperoxide ions ((OOH⁻) produced when using hydrogen peroxide; and/or hydroperoxycarbonate ions (HCO₄⁻) produced when hydrogen peroxide is combined with a carbonate salt. Hydroperoxycarbonate ions (HCO₄⁻) are a much stronger oxidant than hydroxyl ions (OH⁻) or hydroperoxide ions (OOH⁻), and are especially effective in reacting with biological toxants and resistant microorganisms. When using hydrogen peroxide in the inventive embodiments disclosed herein, its concentration is preferably less than about 10% because higher concentrations are significantly corrosive, especially in the range of 30-50% hydrogen peroxide concentration.

When present in compositions according to the instant invention, preferred levels of hydrogen peroxide or equivalent active oxygen, are typically, based on a percent active basis, between 1 to 10 wt. %, or alternatively between 1 to 8 wt. %, or alternatively between 2 to 6 wt. %, or yet alternatively between 3 to 5 wt. % to insure efficacy of action but prevent undesirable corrosivity effects associated with hydrogen peroxide solutions having greater than about 10 wt. % active oxygen. An added advantage of commercial products formulated with below 8 to 10 wt. % of hydrogen peroxide includes less restriction on shipping and storage owing to the US Department of Transportation (DOT) regulations with regard to strong oxidants.

### Stabilizer

Suitable stabilizers for use in embodiments of the inventive compositions include chelants, sequestrants and related builders that are capable of binding trace metal contaminants that may otherwise be detrimental to oxidant stability, and in particular hydrogen peroxide and oxidative by-products thereof. Suitable sequestrants include organic phosphonates, alkanehydroxyphosphonates and carboxylates available under the DEQUEST trade mark from Thermphos and other suppliers.

For example, but not limited to this example, embodiments of the instant invention can use 1-hydroxethane-1,1-diphosphonic acid (HEDP) [CAS 2809-21-4] available under the tradename Dequest 2010 from Italmatch USA Corporation (660 White Plains Rd, Suite 510, Tarrytown, N.Y. 10591 USA) or under the same brand name from UNIVAR/ThermPhos Company (7425 E. 30^{th} St., Indianapolis, Ind. 46219 USA). Another suitable stabilizer is Mayoquest 1500, HEDP, 60% available from Compas Chemical International, LLC (Smyrna Plant, 5544 Oakdale Road, SE, Smyrna, Ga. 30082 USA)

In other embodiments of the instant invention, suitable stabilizers further include, but are not limited to, ATMP (Dequest 2000; amino-trismethylenephosphonic acid), Dequest 2060 (Diethylenetriamine penta(methylene phosphonic acid), Dequest® 2066 (Diethylenetriamine penta(methylene phosphonic acid or Heptasodium DTPMP). In other embodiments of the instant invention, aminocarboxylate sequestrants can be suitable employed, including for example, but not limited to, ethylenediaminetetraacetic acid (EDTA), N-hydroxyethylene-diaminetetraacetic acid (HEDTA), nitrilotriacetic acid (NTA), N-hydroxyethyl-aminodiacetic acid, diethylene-triaminepentaacetic acid (DTPA), methylglycinediacetic acid (MGDA), and alanine-N,N-diacetic acid.

In various embodiments of the instant invention, the level of stabilizer used is typically, based on an active weight %, between 0.030 wt. % to 0.60 wt. %, or alternatively between 0.035 wt. % to 0.55 wt. %, or alternatively between 0.04 wt. % to 0.50 wt. %, as needed with respect to the selected level of hydrogen peroxide or active oxygen present in the final composition.

As will be discussed hereinbelow, there appears to be an optimal range of stabilizer indicated for use in embodiments of the instant invention dependent on the level of active oxygen or hydrogen peroxide present.

### pH Adjuster

Suitable acids and bases for use in adjusting the initial pHs of embodiments of the inventive compositions include for example, but are not limited to, concentration bases such as 50% sodium hydroxide (NaOH) solution or 50% potassium hydroxide (KOH) solution, both commonly available from a host of chemical suppliers, sodium carbonate (anhydrous, 100% active) from Brenntag Corporation (3111 N Post Rd., Indianapolis, Ind. 46226), sodium silicate solutions, commonly available from a host of chemical suppliers. Most preferred are materials that can adjust the pH of aqueous solutions of the inventive compositions without providing a strong buffering effect, which at the levels of addition needed does not place any particular limit on the materials selected. Other suitable materials include concentrated acids such as, but not limited to, phosphoric acid, sulfuric acid and sulfonic acids. In general, only trace amounts of a pH adjuster are necessary, if at all, for adjusting the pH of embodiments of the instant invention.

In respective embodiments of the instant invention, the amount of pH adjusting material required depends on the intermediate pH of the composition after all other ingredients have been added together, followed by adding a sufficient quantity of a suitable base to increase the composition pH to a final desired value, or by adding a sufficient quantity of a suitable acid to decrease the composition pH to a final desired value.

Typically, the amount of adjusting acid or base, particularly if using a highly concentrated or pure form of material, is very small, generally only a drop or two needed in a 1 kg weight aliquot of composition in order to achieve the desired pH adjustment.

### Water

Since the composition is an aqueous composition, water can be, along with the solvent, a predominant ingredient. The water should be present at a level of less than 99 wt. %, or alternatively less than about 98 wt. %, or alternatively, less than about 97 wt. %, or yet alternatively less than about 96 wt. %. The water may be deionized, industrial soft water, or any suitable grade or water. Preferred is water having the lowest content of any contaminating metals or metal cations that can destabilize the hydrogen peroxide material.

### Compositions

Various approaches were explored to enhance hydrogen peroxide stability for the purposes of obtaining compositions exhibiting desirable extended storage capability without loss of activity, while simultaneously providing for enhanced biocidal activity when the inventive compositions are used, following application to a surface with a biological or other contaminant present thereon that is desired to be destroyed or neutralized.

Without being bound by theory, it is believed that the stabilizers normally selected for use with active oxygen systems containing or generating hydrogen peroxide operate in part by providing some degree of pH buffering effect, in addition to their primary role of sequestering metal cations that are known in the art to destabilize peroxide by catalyzing autotropic self-decomposition of the active oxygen species present and produced in the presence of peroxide sources, including molecular hydrogen peroxide, superoxide anion radicals (.O₂⁻), hydroxyl radicals (.OH), hydroxyl ions (OH⁻), hydroperoxide ions (OOH⁻) and/or hydroperoxycarbonate ions (HCO₄⁻), the latter being produced when hydrogen peroxide is combined with a carbonate salt. Hydroperoxycarbonate ions (HCO₄⁻) are a much stronger oxidant than hydroxyl ions (OH⁻) or hydroperoxide ions (OOH⁻), and the like, but all of the above are highly reactive intermediates and thus short lived in aqueous solutions.

Maintaining pH is important for storage stability of hydrogen peroxide solutions in order to preserve the compositions against the destabilizing effects of time, temperature, and storage conditions, such as exposure to ambient light which may contain ultraviolet components, that all act together to reduce peroxide content, resulting in undesirable decreased efficacy of action at time of use. Accordingly, lower pH ranges of between about pH 1.5 to about pH 2.5 are most desirable for longer term stability, pH 2.0 to about pH 3.0 is also suitable, and between pH 2.5 and 3.5 also suitable for hydrogen peroxide solutions, particularly in the absence of other ingredients or contaminants. When other materials are present, lower acidic pH's are generally preferred, sometimes requiring adjustment of the solution pH owing to changes induced by introduction of the other components that have differing pH's and or contribute to a pH change through a dilution or buffering effect.

However, aqueous hydrogen peroxide is more reactive at higher pHs, generally above pH 3.5 where its oxidation potential, and hence efficacy against microorganisms and toxants is accordingly higher owing to the increased solution activity at elevated pHs resulting from the production of one or more of the reactive species disclosed above. Storage of peroxide solutions at higher pH's however has the destabilizing effect on subsequent activity, particularly at storage times up to, and including a year, which is often regarded as a minimum storage time for registered disinfectant products whose activity is desirous of being maintained at initial levels for efficacy of action when needed. While traditional approaches have been to separate the peroxide component from other actives and components in dual packaging or dual delivery systems where the resulting treatment composition is formed instantly in situ by the combination of the two separated components, this approach requires additional packaging, mixing steps, and time to employ, and is subject to error in application owing to the necessary mechanical components and mixing otherwise required. Thus, it is highly desirous to employ hydrogen peroxide based disinfectants, particularly those including other active biocides, in a single, ready to use solution that requires no special packaging, bottling, delivery means, or mixing steps prior to use.

Accordingly, without being bound by theory, it has been discovered that the proper selection of initial pH, the amount of stabilizer, and the elimination of buffering species in hydrogen peroxide based disinfectant solutions can result in the retention of desired peroxide stability, while enabling the delivered efficacy of the peroxide species by providing the means for it to become activated by a pH change upon application to a surface at time of use.

Without being bound by theory, it is believed that when hydrogen peroxide solutions are applied to a surface, such as for example by depositing a liquid film either by submersion, spraying a solution thereon, or applying a solution by means of a saturated wipe, a thin aqueous liquid film is deposited on that surface and immediately experiences subsequent changes in nature relating to its pH and composition that are exploited by means of the present inventive formulations. For example, after application to a surface, the thin aqueous liquid film immediately begins to evaporate due to loss of water, hydrogen peroxide and other volatile chemical components present, which acts to concentrate other actives and hydrogen peroxide as well, owing to the slightly lower vapor pressure of the peroxide species compared to water, as hydrogen peroxide has a slightly higher molecular weight than water and additional hydrogen bonding interactions with the polar continuous water solvent that slightly favor its retention in solution versus a water molecule. In addition, atmospheric exposure to air, which contains carbon dioxide, results in some absorption of this gas by the aqueous liquid film, this gas being highly soluble in water and which, when absorbed and solubilized therein, results in the production of free carbonate and bicarbonate anions, depending on the initial and then the changing, instantaneous pH of the thin film as atmospheric exposure continues. The absorption of carbon dioxide results in a change of pH, the nature of carbonate/bicarbonate equilibrium contributing to a buffering effect around the natural pKa (log Ka or logarithm of the equilibrium constant) of carbon dioxide solubilized in water. The pKa of the carbonate system is approximately pH 5.5, so that with sufficient time, an aqueous solution of water, and in particular a thin film with high surface area exposed to ambient carbon dioxide, would be expected to continue to absorb available carbon dioxide until an equilibrium state is reached and a nominal solution pH approaching pH 5.5 is achieved. Naturally, other reactive species participate in the other equilibrium state and effect to control the overall pH, but in operation a rise in the pH of a thin aqueous film of solution is expected to occur unless the initial aqueous solution is heavily buffered to prevent subsequent pH changes. The absorption of carbon dioxide is fairly efficient and continues until the applied aqueous liquid film has either fully evaporated or has been removed from the surface by a subsequent wiping or removal mechanism. In addition, it is believed that the nature of surface contaminants themselves, and particularly biological fluids and residues, tend to have higher pHs then the inventive compositions described herein, which also contribute to the ability of embodiments of the instant invention to undergo an increase in pH following application to a surface bearing those contaminants, helping the hydrogen peroxide component to autoactivate and demonstrate enhanced or accelerated biocidal and oxidative efficacy owing to the higher pH and increased oxidation potential.

In the inventive hydrogen peroxide-based compositions disclosed herein, it has been discovered that by careful selection of, and reduction of the amount of stabilizer present, that both desired storage stability and ultimate efficacy of action may simultaneously be achieved. Without being bound by theory, it is believed that the embodiments of the inventive formulations disclosed herein are capable of maintaining storage stability over long periods of time by means of a suitable level of a sequestrant that has minimal or no buffering capability, and that this minimal or lack of buffering capability, in the absence of other buffering materials, enables the thin, applied films of the inventive compositions to auto-activate when applied to surfaces by means of enabling the pH of the resulting thin films to increase following contact with the surface, with surface contaminants thereon, and with continued atmospheric absorption of carbon dioxide sufficient over the exposure or contact time to provide for an increased activity of peroxide and other generated reactive oxygen species as disclosed herein. Thus, by the instant inventive means of selecting stabilizers and their levels at appropriate levels so as to provide for storage stability but not to result in over-stabilization or buffering of the applied solutions, inventive embodiments of the instant invention are enabled to undergo a transition at the surface from their nominal pHs to subsequently higher pHs, resulting in a significantly enhanced biocidal effect as well as improved oxidation of toxants present on surfaces treated with embodiments of the instant invention in the form of compositions applied as thin aqueous films thereon.

To explore the amount of optimum sequestrant or stabilizer required to provide for sufficient or minimum storage stability for the inventive compositions, a series of fourteen (14) test compositions employing only the selected stabilizer and hydrogen peroxide were prepared and evaluated under various storage conditions and at time intervals of approximately one month for up to twelve (12) months of storage.

To prepare the test compositions described above, stock solutions of peroxide were prepared using Interox U.P. 50% hydrogen peroxide, and deionized water to produce diluted peroxide compositions having a nominal initial concentration of 7.0 wt. % hydrogen peroxide. Individual solutions were prepared next, starting with exactly 100 g of the 7% peroxide solution to which the stabilizers are then added to achieve the amounts indicated in Table 1. In these embodiments of the instant invention, Dequest 2010 (HEDP) was used. The Dequest 2010 wt. % values are also shown in the table, as well as the final pH's of the individual test compositions after the completion of the one year storage test. Note that a level of 0.08 wt. % HEDP is equivalent to about 480 ppm, and a level of 0.12 wt. % HEDP corresponds to about 720 ppm present.

In Table 1, compositions 1, 2 and 3 are control samples, designated as Group 1, being a formulated hydrogen peroxide disinfectant product, SpectraKill SK-101, available from Spectra Shield Technologies (3610 North 44th St #130, Phoenix, Ariz. 85018), providing three replicates selected to represent the current state of the art with respect to commercial stabilized hydrogen peroxide disinfectants. Samples were selected or prepared to have essentially equivalent starting levels of hydrogen peroxide, and where indicated, adjusted by the addition of a concentrated acid or base to have an essentially equivalent starting pH level, being approximately pH 2.2

Test compositions 4-6 represent embodiments of the instant invention which together with the control composition 7, designated Group 2, were formulated and exposed to benchtop room temperate conditions whereby the test compositions were exposed to ambient light including nominal ultraviolet light present comparable to that found in a residential, commercial, industrial, or medical environment having overhead fluorescent lighting installed. Test composition 4 contained 0.08 wt. % Dequest 2010, while test composition 5 contained a higher 0.12 wt. % level of Dequest 2010, the latter requiring a slight pH adjustment obtained by the drop wise addition of a sufficient amount of 50% caustic soda (sodium hydroxide solution). Test composition 6 is a control sample without Dequest stabilizer, but whose pH was adjusted using the drop wise addition of a sufficient amount of 75% phosphoric acid. Further, test composition 7 is a control sample where no initial pH adjustment was made using either caustic or acid, to show the properties of a non-pH adjusted, or unmodified diluted hydrogen peroxide composition.

Test compositions 8-11, designated Group 3, where formulated as indicated and tested after storage in the dark at ambient room temperature (average, approximately 25° C.) so that the effect of light exposure on ultimate peroxide stability could be assessed. Test composition 8 is a control solution corresponding in formulation to test composition 6, while test composition 10 is a control solution corresponding in formulation to test composition 7, the latter of each set differing only in storage conditions as indicated. Test compositions 9 and 11 are representative embodiments of the invention corresponding to test compositions 4 and 5, respectively, the latter of each set differing only in storage conditions as indicated, as well.

Test compositions 12-14, designated Group 4, where formulated as indicated and tested in the dark but at an elevated storage temperature of approximately 110° F. (or 43° C.) to accelerate the effect of storage temperature. Test composition 12 is a control solution corresponding to test compositions 6 and 8, but with different storage conditions. Test compositions 13 and 14 are representative embodiments of the instant invention that correspond to compositions 4 and 8, and 5 and 11, respectively. Further, Table 1 shows the initial, optionally, adjusted pH values of the solutions, taken at the start of the test.

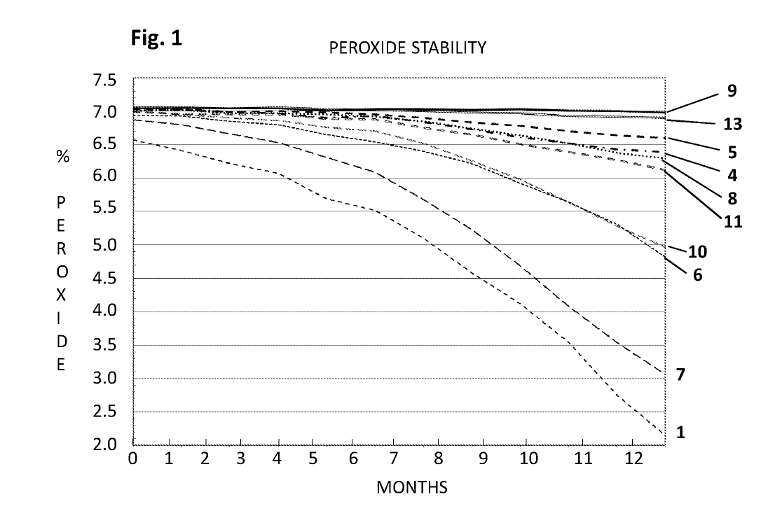

The respective stability trends of the test compositions detailed in Table 1 are more easily interpreted by referring to FIG. 1, which shows the remaining % hydrogen peroxide activity derived by suitable titration of an aliquot of the test compositions taken at the time interval indicated, plotted as a function of storage time. The traces that are numbered and shown in FIG. 1 correspond to the instantaneous assayed level of hydrogen peroxide measured for the respective controls, comparative samples and inventive embodiments as shown in Table 1.

In FIG. 1, it is seen that the representative test samples of Group 1, corresponding to a commercial product, SpectraKill SK-101, exhibit poor stability over time in a benchtop configuration exposed to room light and ambient ultraviolet (UV) radiation present therein. In contrast, embodiments of the instant invention represented by test compositions 4 and 5 of Group 2 exhibit very good stability under the same challenge conditions, and also as compared to the Group 2 control test composition 6, which was a pH adjusted peroxide solution without any stabilizer present and which performed poorly, being only slightly better in long term stability than the commercial test product represented by comparative test composition 1.

Further, in FIG. 1 the contrast in stability trends between test compositions stored under ambient light and dark (no light) conditions can clearly be seen, test composition 10 having a much improved, but still somewhat unacceptable hydrogen peroxide stability when stored in the dark as compared to the equivalent composition after light exposure (test composition 7), the latter failing to meet the desired stability level.

With respect to the level of stabilizer, embodiments of the instant invention corresponding to test compositions 4, 9 and 13 all employed the lower level of 0.08 wt. % Dequest 2010, while test compositions 5, 11 and 14 (not shown) all employed a higher level of 0.12% Dequest 2010. It can be seen that compositions employing the lower level of stabilizer tend to significantly outperform similar compositions having a higher level of the same stabilizer, even though the overall stability of both sets of compositions are acceptable. This is readily seen by comparing inventive embodiments corresponding to test composition 5 vs 4 under ambient lighting conditions, and the similar set of compositions stored in the dark at room temperature (RT) corresponding to the inventive embodiment compositions 9 vs 11. Under both RT storage conditions either including light and ambient UV, or storage in the dark, embodiments of the instant invention employing the lower level of stabilizer generally outperformed those with a higher level of stabilizer. This same trend is confirmed under stress storage conditions of elevated temperature seen by contrasting the stability of the inventive embodiment test composition 13 with the lower stabilizer (Dequest 2010) level, compared to test composition 14 with the higher level, and the control test composition 12 (not shown in FIG. 1), whose ultimate hydrogen peroxide levels dropped below 2% at the conclusion of the test. (See Table 1).

Accordingly, it can be concluded that an optimum range of stabilizer exists, and that too low or alternatively too high an amount of stabilizer can result in unacceptable storage stability when used in conjunction with an aqueous active oxygen containing composition, particularly one containing molecular hydrogen peroxide. This is in contrast to conventional wisdom that would predict that moderately increasing amounts of stabilizer tend to increase storage stability and that even though the effects may eventually tend to level off, increasing concentration of stabilizer merely providing little additional benefit, that no significantly detrimental impact of reasonably higher levels (such as an additional aliquot or mole fraction) is anticipated. In sharp contrast, the instant discovery here clearly demonstrates the unexpected result that even slightly higher amounts of stabilizer outside of the inventive range are in fact detrimental to storage stability, and tend to decrease stability performance after some critical level of stabilizer is passed, and accordingly impact the subsequent efficacy at the desired time of use of the active oxygen containing compositions of the instant invention as embodied herein.

Considering the results of the stability testing of hydrogen peroxide solutions shown in Table 1 and in FIG. 1, there appears to be an optimum range of stabilizer for stabilizing the pH of embodiments of the instant invention, with some upper limit providing sufficient RT and elevated storage temperature stability, yet not interfering with the auto-activation of the hydrogen peroxide when it is eventually applied in the form of an inventive composition onto a surface as a thin, aqueous film. As stabilizer levels increase, the buffering nature of the materials is believed to interfere with the auto-activation process by over-stabilizing the pH when it is actually desirable to allow the inventive compositions, when present as thin, aqueous films to undergo a pH increase from interacting with the surface, microbes and toxants present, and carbon dioxide present in the atmosphere.

Accordingly, it has been found that a preferred ratio of hydrogen peroxide to stabilizer, expressed as a ratio of the two materials expressed as actives in weight %, or (Peroxide, wt. %)/(Stabilizer, wt. %) (“P:S Ratio”) is indicated for use in embodiments of the instant invention. The P:S Ratio preferred for use herein for embodiments of the instant invention typically range from between 150:1 to 125:1, or alternatively from between 125:1 to 100:1, or alternatively from between 110:1 to 95:1, or yet alternatively from between 100:1 to 90:1. Representative compositions corresponding to embodiments of the instant invention are also shown below in Table 1.

TABLE 1

| | Solution | | | | | | | | | | | | | |
|---|---|---|---|---|---|---|---|---|---|---|---|---|---|---|
| Time | 1 | 2 | 3 | 4 | 5 | 6 | 7 | 8 | 9 | 10 | 11 | 12 | 13 | 14 |
| (Days) | % Peroxide | | | | | | | | | | | | | |
| 29 | 6.57 | 6.51 | 6.64 | 7.05 | 7.03 | 6.94 | 6.87 | 7.01 | 7.04 | 7 | 6.99 | 7.01 | 7.06 | 6.9 |
| 36 | 6.41 | 6.32 | 6.48 | 7.03 | 7.02 | 6.93 | 6.8 | 7.02 | 7.05 | 6.95 | 6.96 | 6.9 | 7.06 | 6.77 |
| 43 | 6.22 | 6.19 | 6.32 | 6.99 | 6.99 | 6.85 | 6.66 | 6.97 | 7.04 | 6.9 | 6.96 | 6.76 | 7.04 | 6.63 |
| 50 | 6.07 | 5.92 | 6.17 | 6.97 | 7 | 6.8 | 6.53 | 6.97 | 7.04 | 6.86 | 6.95 | 6.7 | 7.06 | 6.49 |
| 64 | 5.69 | 5.65 | 5.74 | 6.9 | 6.98 | 6.65 | 6.31 | 6.95 | 7.01 | 6.76 | 6.89 | 6.31 | 7.03 | 6.14 |
| 78 | 5.52 | 5.42 | 5.48 | 6.93 | 6.95 | 6.54 | 6.09 | 6.92 | 7.03 | 6.7 | 6.88 | 5.84 | 7.02 | 5.92 |
| 99 | 5.1 | 4.95 | 5.14 | 6.84 | 6.9 | 6.4 | 5.68 | 6.85 | 7.03 | 6.52 | 6.75 | | 7 | |
| 141 | 4.58 | 4.55 | 4.78 | 6.73 | 6.83 | 6.22 | 5.23 | 6.74 | 7.02 | 6.26 | 6.65 | | 6.9 [symbol] | |
| 197 | 4.12 | 4 | 4.28 | 6.61 | 6.78 | 5.92 | 4.69 | 6.64 | 7.03 | 5.98 | 6.51 | | 6.97 | |
| 260 | 3.54 | 3.32 | 3.75 | 6.52 | 6.7 | 5.64 | 4.08 | 6.52 | 7.01 | 5.64 | 6.39 | | 6.93 | |
| 351 | 2.75 | 2.59 | 3.2 | 6.43 | 6.64 | 5.31 | 3.54 | 6.38 | 7 | 5.28 | 6.27 | 2.01 | 6.92 | 2.03 |
| 386 | 2.15 | 2.05 | 2.63 | 6.39 | 6.6 | 4.82 | 3.0 [symbol] | 6.29 | 6.98 | 4.97 | 6.12 | 1.34 | 6.9 | 1.95 |
| pH | 3.56 | 3.68 | 3.48 | 2.2 | 2.17 | 2.11 | 3.72 | 2.15 | 2.17 | 3.72 | 2.02 | 2.13 | 2.14 | 2.02 |
| Storage | Benchtop | Benchtop | Benchtop | Benchtop | Benchtop | Benchtop | Benchtop | Dark, | Dark, | Dark, | Dark, | Dark, | Dark, | Dark, |
| | | | | | | | | RT | RT | RT | RT | 110° F. | 110° F. | 100° F. |

Note:
Initial pH measured at start of study. Storage conditions were one of: Benchtop (exposed to light and UV) or Dark at room temperature, or Dark at elevated temperature. [symbol]
indicates data missing or illegible when filed

### Sample Preparation

Formulations relating to the inventive compositions are readily prepared by means of the following sample preparation steps, and while these steps can be prepared in different order, it is usually preferred to start with the largest diluent being present in the mixing vessel selected, being water, followed by addition of any stabilizer components. Accordingly, the following preparation steps were used in preparing embodiments of the invention, starting with (1) charging purified water to an appropriate mixing vessel; (2) agitating the solution and adding stabilizer (Dequest 2010) to the vessel; (3) adding hydrogen peroxide with continued agitation followed by; (4) addition of the second biocidal material, quaternary ammonium biocide (Maquat MC 1412-50) with agitation and then: (5) mixing all components with continued agitation until the resulting solution is completely clear and uniform, and any foam, if present, is reduced or reconstituted back into solution.

Example embodiments of the inventive compositions prepared, along with acceptable variations in the ranges of the components expected under manufacturing conditions and variability associated therewith, are shown below in Table 2.

TABLE 2

| | | Percent | | | Weight | | Activity |
|---|---|---|---|---|---|---|---|
| | Activity | By | | | Limits | Activity | Limits |
| Component | (%) | Weight | Amount (5) | | (wt %) | (wt %) | (wt %)(6) |
| Water (1) | 100 | 89.72 | 897.2 | g | 87.03-92.41 | q.v. | q.v. |
| Dequest 2010 (2) | 60 | 0.08 | 0.8 | g | 0.07-0.09 | 0.048 | 0.042-0.054 |
| Hydrogen Peroxide (3) | 50 | 10.0 | 100.0 | g | 9.5-10.5 | 5.0 | 4.75-5.25 |
| Maquat MC1412 (4) | 50 | 0.2 | 2.0 | g | 0.18-0.22 | 0.1 | 0.09-0.11 |

(1) Deionized or distilled water.
(2) Dequest 2010, being 60 wt. % HEDP, from Italmatch.
(3) Hydrogen Peroxide B-Cap 50 Antibacterial, from PeroxyChem.
(4) Quaternary ammonium chloride, Maquat MC 1412-50, from Pilot Chemical Company.
(5) Amounts sufficient to prepare approximately 1 kg of solution.
(6)Activity, expressed in actual amount of active present in wt. %. Note that water totals generally reflect included water or other diluents (solvents) present in the other raw materials, quantity sufficient (q.v.) for 100% activity with respect to total water content.

In the various embodiments of the instant invention, the formulations produced had initial pH's in the range of between pH 2 and pH 3, with specific gravity (density as compared to distilled water) of between about 1.01 and 1.03. In these embodiments of the instant invention, the resulting hydrogen peroxide concentrations or equivalent amount of active oxidant present expressed in terms of free hydrogen peroxide generally ranged from between 3 to 6 wt. %, or alternatively from 3.5 to 5.5 wt. % or alternatively from 4.0 to 5.0 wt. %. Further, in these representative embodiments of the instant invention, the resulting quaternary biocide concentrations generally range, in terms of ppm, from between 500 to 2000 ppm, or alternatively from between 750 to 1500 ppm, or alternatively from between 850 to 1250 ppm.

### Storage Stability

The storage stability of several embodiments of the inventive compositions were evaluated by submitting samples to Case Laboratories, Inc. (622 Route Ten, Whippany, N.J. 07981 USA) to determine whether the compositions had the requisite shelf stability to maintain efficacy over prolonged time and exposure conditions, in compliance with 40 CFR § 158.310: Product Chemistry Data Requirements Table OPPTS Test Guidelines, Series 830, Product Properties, Group B—Physical/Chemical Properties: 830.6317 and 830:6320, respectively.

One lot (032316-RD) of the inventive composition corresponding to Composition 25 (see Table 11) was selected having a nominal hydrogen peroxide concentration of 5.35 wt. % as active, and an alkyl ammonium cationic biocide concentration of 0.117 wt. % as active.

The test was conducted by contacting the test composition with commercial packaging material, being a high-density polyethylene (HDPE) bottle. Evaluations were conducted at study initiation and after fourteen (14) days at accelerated storage conditions of 54° C.±2° C., using methods described hereinbelow. At least two replicate analyses were performed at each interval.

### Determination of Quaternary Amine

Reference materials used included 0.1 N ceric sulfate solution, Lot no.: Z04B066, obtained from Alfa Aesar (2 Radcliff Rd., Tewksbury, Mass. 01876) and benzethonium chloride (98.8% activity), Lot No.: 2DK0233, obtained from Spectrum Chemical (769 Jersey Avenue New Brunswick, N.J. 08901-3605).

Reagents included (a) 50% sodium hydroxide obtained by mixing equal weights of sodium hydroxide and deionized water; (b) sodium lauryl sulfate (SLS), 0.003N obtained by adding approximately 1.5 to 1.8 g of SLS into a 2000 mL volumetric flask followed by addition of 2 drops of 50% sodium hydroxide and bringing the total volume to 2000 mL using distilled water and later standardized against the benzethonium chloride reference solution; (c) chloroform, reagent grade; (d) salt buffer solution obtained by combining 14 g of sodium carbonate and 200 g of sodium sulfate and diluting to 2000 mL using distilled water; (d) bromophenol blue indicate solution obtained by preparing a 0.10 wt. % solution of the indicator in deionized water; and (e) deionized water.

SLS was first standardized by following these steps: (a) drying the benzethonium chloride standard for 1 hour at 105° C.; (b) accurately weighing 100 mg of dry benzethonium chloride (BZC) into a 100 mL volumetric flask and diluting to volume using deionized water; (c) pipetting 10 ml of the preceding solution into a 250 mL iodine flask; (d) adding 50 mL chloroform, 50 mL of the salt buffer solution and 6 drops of bromophenol blue indicator to each flask; (e) stoppering the flasks and shaking vigorously; and then (f) titrating the benzethonium chloride with aliquots of 0.003 N SLS, shaking vigorously between additions, the end point being determined at the first definite (persistent) appearance of a violet color in the upper layer. The standardization was performed with at least three replicates.

The normality of the sodium lauryl sulfate solution is calculated according to the following equation:

$$N = \frac{\begin{matrix}( g BZC ) ( Aliquot size , 10 mL ) \\ ( 1000 meg \text{/} eq ) ( Percentage purity of BZC )\end{matrix}}{( 448.1 g \text{/} meq ) ( Titer , mL ) ( Total Volume , mL )}$$

wherein the molecular weight (MW) of benzethonium chloride is 448.1 g/mol.

Following standardization, the following assay procedure was followed, starting by (a) accurately weighing approximately 18 g of test substance into 250 mL iodine flasks; (b) adding 50 mL of chloroform, 50 mL of salt buffer solution and 5-8 drops of bromophenol blue indicator to each flask; (c) stoppering the flasks and shaking the solutions vigorously; and (d) then titrating with standardized 0.003N sodium lauryl sulfate, shaking vigorously between additions, the end point being determined by the first definite appearance of a violet color in the upper layer. The assays were performed with at least three replicates.

The percent of active quaternary amine (QA) material in the test substances is then calculated using the following equation:

$$\% Quaternary Amines = \frac{( mL SLS ) ( Normality SLS ) ( M W )}{( Sample weight , g ) ( 10 )}$$

wherein the molecular weight (MW) of quaternary amines is 358 g/mol.

### Determination of Hydrogen Peroxide

Reagents used to quantitate peroxide levels include 1N sulfuric acid, ferroin indicator solution and 0.1N Ceric Sulfate, standardized, which can all be obtained from any chemical supply company. The peroxide assay was then conducted by following the steps of (a) accurately weighing approximately 0.5 g of the test substance into a 125 mL Erlenmeyer flask; (b) adding 50 mL of cold (refrigerated or previously placed in a water-ice bath) 1N sulfuric acid; (c) adding 4 drops of ferroin indicator; (d) stirring the solution using a magnetic stir bar (Teflon coated); and (e) then titrating with 0.1N ceric sulfate until a light blue color is observed denoting the endpoint.

Calculation of the concentration of hydrogen peroxide in the sample is then obtained using the following equation:

$$\% Hydrogen Peroxide = \frac{( Titer , mL ) ( Normality Ceric Sulfate ) ( 17.01 )}{( Sample weight , g ) ( 1000 mL \text{/} L )} \times 100$$

### Physical Changes

At the 14-day storage stability interval, the test substances are examined for physical changes such as color, phase separation, precipitation, clumping, and, in particular, any variations that would interfere with the usefulness or safe handling of the product if used according to label directions.

### Corrosion Characteristics

At the 14-day storage stability interval, the commercial packaging materials, HDPE, used for the storage stability test was visually inspected, and any changes in the material were noted and recorded.

Results show that the inventive compositions have the requisite storage stability and are absent any observed corrosivity suitable for commercial product usage and in compliance with storage longevity and efficacy requirements of the United States Environmental Protection Agency (US-EPA).

The storage stability of the two active materials, quaternary amines and hydrogen peroxide were found acceptable, showing essentially no significant change in activity levels for either active material present in the inventive compositions, as noted in the summary Table 3 below:

TABLE 3
Storage Stability:
% Quaternary Amines

| Interval | Rep. 1 | Rep. 2 | Rep. 3 | Rep. 4 | Average |
|---|---|---|---|---|---|
| Initial | 0.119% | 0.120% | — | — | 0.120% |
| After 14 Days at | 0.121% | 0.123% | 0.120% | 0.119% | 0.121% |
| 54° C. ± 2° C. | | | | | |

% Hydrogen Peroxide

| Interval | Replicate 1 | Replicate 2 | Average |
|---|---|---|---|
| Initial | 5.32% | 5.35% | 5.33% |
| After 14 Days at | 5.71% | 5.38% | 5.54% |
| 54° C. ± 2° C. | | | |

| | |
|---|---|
| Interval | Observation |

Physical Changes:

| | |
|---|---|
| After 14 Days at | No physical changes were noted. |
| 54° C. ± 2° C. | |

Corrosion Characteristics

| | |
|---|---|
| After 14 Days at | No corrosion present on commercial |
| 54° C. ± 2° C. | packaging material, HDPE |

### Efficacy Testing

To ensure that embodiments of the instant invention have the desired antimicrobial efficacy in addition to the enhanced storage stability obtained from optimizing the choice of stabilizer and relative stabilizer concentration, test compositions were prepared and sent to Microchem Laboratory (located at 1304 W Industrial Blvd., Round Rock, Tex. 78681 USA) for efficacy testing against two critical microorganisms following the EPA test procedure qualified under ASTM E1153-NG7458 standard methodology.

Results of the test are shown in Table 4 for two microorganisms, Escherichia coli (ATCC® 8739™) and Staphylococcus aureus subspecies aureus (ATCC® 6538™), which are commonly found in hospital environments and responsible for hospital acquired infections amongst patients and staff members due to their antimicrobial resistance.

TABLE 4

| | | | | Percent Reduction | Log₁₀ Reduction |
|---|---|---|---|---|---|
| Test | Contact | Carrier | | Compared to Control | Compared to Control |
| Microorganism | Time | Type | CFU/Carrier | at Contact Time | at Contact Time |
| E. Coli | 30 Seconds | Control | 7.88E+05 | N/A | |
| 8739 | | C61716MM | <1.25E+01 | >99.998% | >4.80 |
| S. aureus | | Control | 1.65E+07 | N/A | |
| 6538 | | C61716MM | 1.18E+03 | 99.99% | 4.15 |

In efficacy testing, the microorganisms are applied to a suitable media in accordance with the ASTM methodology in an amount equivalent to about 8×10⁵ CFU/mL for E. Coli, and about 2×10⁷ CFU/mL for S. aureus. A Colony Forming Unit (CFU) is defined as the minimum number of organisms required to establish a viable colony on the selected media, having the ability to reproduce and sustain itself thereon, corresponding to a minimum population that would be sufficient for reproduction of the microorganism colony. Not surprisingly, a single drop of infected biological fluid such as blood, tears, puss, sweat or other bodily secretions can have as much or even multiple magnitudes of order (factor of 10 per order) greater amounts of the microorganism present, accounting for the rapid spread and colonization of these types of virulent bacteria in human environments and within humans and other mammals susceptible to their infective natures. For microbial testing, a contact time of 30 seconds was selected, being a reasonable time for application of a disinfectant to a surface to be treated. Dual control samples were included, and were found to have no significant reduction in microbial count, verifying that the test conditions were not responsible for any observed kill or reduction in population or colony counts.

For E. Coli, the test sample embodiment was effective at reducing the initial CFU count to below the detection limit, or essentially to zero, corresponding to the reported log₁₀ reduction of greater than (>) 4.80, showing a reduction of greater than 99.998% of the population of that bacterial species on the C61716MM carrier, which is a standard having all the required nutrients and growth factors otherwise present in biological tissue as needed for bacterial growth and reproduction, as shown in Table 4.

For S. aureus, the test sample embodiment was effective at reducing the initial CFU count to the reported log₁₀ reduction of about 4.15, showing a reduction of greater than 99.993% of the population of that bacterial species on the C61716MM carrier, leaving only a non-viable level of S. aureus corresponding to about 1.18×10³ CFUs.

Note that in Table 4, the limit of detection is approximately 12.5 Colony Forming Units (CFU)/mL for the E. Coli species, and results are recorded as less than (<) 1.25E+01, which is essentially equivalent to the detection limit within one standard deviation, and thus essentially being zero.

Thus, microbial reduction tests show that the inventive compositions embodied here are quite effective in their antimicrobial efficacy while simultaneously exhibiting extended storage stability under both typical and stress conditions of heat and light exposure. This combination of factors provides the embodiments of the instant invention with the highly advantageous properties needed in a commercial product whose storage stability and efficacy must be maintained for the compositions to continue to exhibit their desirable properties for use in hospitals and other demanding environments where stopping the growth and spread of microorganism is essential for human health.

Further testing on other microorganisms was completed as well to demonstrate the effectiveness of various embodiments of the instant invention to kill germs efficiently.

### AOAC Germicidal Spray Test

Originally developed in 1961, the AOAC (Association of Official Agricultural Chemists) Germicidal Spray Test is a standard method for evaluating the efficacy of liquid disinfectants on hard, non-porous surfaces. It is one of the EPA-specified test methods for antimicrobial pesticide registration. This method is particularly appropriate for disinfectants which do not require dilution and are designed to be used with an aerosol or trigger spray. The method is semi-quantitative, meaning that it relies on a series of qualitative observations (a test tube changing from clear to turbid as a result of microbial growth).

The above test was conducted by MicroBioTest, a division of Microbac Laboratories, Inc. (105 Carpenter Drive, Sterling, Va. 20164 USA) on three common microbial species, Staphylococcus aureus (ATCC 6538), Pseudomonas aeruginosa (ATCC 15442) and Salmonella enterica (ATCC 10708). Test methodology corresponded to EPA Guidelines 810.200(D)(1)(ii) and were conducted using a two (2) minute contact time of the test composition with bacteria inoculated onto selected media, for example, a heat-inactivated horse serum providing a 5% organic load, using Letheen broth containing a catalase enzyme as a neutralizer to stop all activity after the proscribed contact time, and conducted at room temperature (20-21° C. at about 47% RH (relative humidity). Media tested in this method (inoculated with the 5% organic serum as load) included synthetic broth (SB), nutrient broth (NB), Neutralizer recovery broth, Letheen Broth (LB), Tryptic Soy Agar (TSA), Mannitol Salt Agar (MSA), MacConkey Agar (MCA) and Xylose-Lysine deoxycholate agar (XLDA).

For evaluation, a one inch square area of each carrier (1″×3″ glass microscope slides) were inoculated with 0.01 mL of the challenge microorganism and dried for 40 minutes at 36° C. and 25-27% RH. Test compositions were applied by spraying the carrier until wet thoroughly, spraying from a distance of between 6 to 8 inches. After spraying or control treatment and a delay period of 2 minutes, the carriers were then placed into tubes containing the neutralizing recovery broth and then evaluated after the proscribed incubation time.

Results are present in Tables 5-7. The challenge microorganisms were confirmed by colony morphology and Gram stain techniques to be consistent with Staphylococcus aureus, Pseudomonas aeruginosa and Salmonella enterica. The sterility control exhibited no growth. The viability and neutralizer effectiveness controls exhibited growth as expected. An evaluation of bacteriostasis was not performed or applicable for this testing.

Table 5 shows test results expressed as the number of tubes exhibiting growth/total number of tubes. Samples were coded into Lot no's for reporting purposes, Lot No. 100213RD-1, 100213RD-2 and 100213RD-3, all identical formulations by composition, and with levels of ingredients corresponding to the inventive embodiment shown in Table 11 as Composition 25.

TABLE 5

[symbol]

| | Lot No. | Lot No. | Lot No. |
|---|---|---|---|
| Microorganism | 100213RD-1 | 100213RD-2 | 100213RB-3 |
| Staphylococcus aureus | 1/60 | 0/60 | 1/60 |
| Pseudomonas aeruginosa | 1/60 | 1/60 | 1/60 |
| Salmonella enterica | 1/60 | 1/60 | 0/60 |

[symbol]

indicates data missing or illegible when filed

Table 6 shows carrier counts obtained expressed as Average Colony Forming Units (CFU) per carrier.

TABLE 6

[symbol]

| | | CFU/carrier | | | Average |
|---|---|---|---|---|---|
| Microorganism | Time Point | Rep 1 | Rep 2 | Rep 3 | CFU/carrier |
| Staphylococcus | Pre-Test | 7.2 × 10⁵ | 9.0 × 10⁵ | 1.3 × 10⁶ | 9.7 × 10⁵ |
| aureus | Post-Test | 1.8 × 10⁶ | 1.9 × 10⁶ | 1.1 × 10⁶ | 1.6 × 10⁶ |
| Pseudomonas | Pre-Test | 9.0 × 10⁵ | 8.4 × 10⁵ | 1.2 × 10⁶ | 9.8 × 10⁵ |
| aeruginosa | Post-Test | 6.2 × 10⁵ | 8.1 × 10⁵ | 6.6 × 10⁵ | 7.0 × 10⁵ |
| Salmonella | Pre-Test | 1.5 × 10⁵ | 1.7 × 10⁵ | 1.5 × 10⁵ | 1.6 × 10⁵ |
| enterica | Post-Test | 1.5 × 10⁵ | 1.3 × 10⁵ | 1.4 × 10⁵ | 1.4 × 10⁵ |

[symbol]

indicates data missing or illegible when filed

Table 7 shows neutralizer effectiveness as either Growth (+) or No Growth (0) and average Colony Forming Units (CFU) per tube as a control to confirm the viability of the bacterial colonies to grow under test conditions without treatment.

TABLE 7

[symbol]

| | | Tube Results | | | | | | Confirmation |
|---|---|---|---|---|---|---|---|---|
| | | Rep | Rep | Rep | Rep | Rep | Rep | Counts |
| Microorganism | Lot Number | 1 | 2 | 3 | 4 | 5 | 6 | CFU/tube |
| Staphylococcus | 100213RD-1 | + | + | + | + | + | + | 34 |
| aureus | 100213RD-2 | + | + | + | + | + | + | |
| | 100213RD-3 | + | + | + | + | + | + | |
| Pseudomonas | 100213RD-1 | + | + | + | + | + | + | 24 |
| aeruginosa | 100213RD-2 | + | + | + | + | + | + | |
| | 100213RD-3 | + | + | + | + | + | + | |
| Salmonella | 100213RD-1 | + | + | + | + | + | + | 52 |
| enterica | 100213RD-2 | + | + | + | + | + | + | |
| | 100213RD-3 | + | + | + | + | + | + | |

[symbol]

indicates data missing or illegible when filed

The above results confirm that the tested compositions all pass the AOAC Germicidal Spray Test, as there was no visible growth observed in at least 59 out of 60 of the subculture broth tubes per lot per microorganism and the controls met their stipulated criteria.

Further AOAC Germicidal Spray Tests were conducted by MicroBioTest on Trichophyton mentagrophytes (ATCC 9533) to measure the effectiveness against this organism, which is a dermatophyte, being a part of a group of three genera of fungi that cause skin disease in people and animals. Test methodology corresponded to EPA Guidelines 810.200(D)(1)(ii) and were conducted using a two (2) minute contact time of the test composition with bacteria inoculated onto selected media, using heat-inactivated fetal bovine serum providing a 5% organic load when added to the inoculum. Tests were conducted at ambient room temperature (20° C.) and at 42-55% RH. Neutralizer broth used was neopeptone glucose broth containing 7% polysorbate 80, 1% lecithin and 0.2% sodium thiosulfate plus catalase. Media tested included Letheen broth and neopeptone glucose agar. Test compositions were sprayed onto the carrier slides (as described hereinabove in prior Spray Test) until thoroughly wet from a distance of between 6 and 8 inches. After the proscribed two (2) minute contact time, the carriers where placed into the neutralization broth and then evaluated after a suitable incubation period.

Results are shown below in Tables 8-10. Table 8 shows test results expressed as the number of tubes exhibiting growth/total number of tubes.

TABLE 8

[symbol]

| | Lot No. | Lot No. |
|---|---|---|
| Microorganism | 102113RD-1 | 102113RD-2 |
| Trichophyton | 0/10 | 0/10 |
| mentagrophytes | | |

[symbol]

indicates data missing or illegible when filed

Table 9 shows results of the test expressed as Average Conidial (Colony) Forming Units (CFU) per carrier.

TABLE 9

[symbol]

| | CFU/carrier | | | Average |
|---|---|---|---|---|
| Microorganism | Rep 1 | Rep 2 | Rep 3 | CFU/carrier |
| Trichophyton | 8.2 × 10⁴ | 9.1 × 10⁴ | 6.4 × 10⁴ | 7.9 × 10⁴ |
| mentagrophytes | | | | |

[symbol]

indicates data missing or illegible when filed

Table 10 shows the neutralizer effectiveness as a control, the results being expressed as Growth (+) or No Growth (0) and the average Colony Forming Units (CFU) per Tube.

TABLE 10
Neutralizer Effectiveness Confirmation
Results Expressed as Growth (+) or No Growth
(0) and Conidial Forming Units (CFU)

| | Tube Result | | Confirmation counts |
|---|---|---|---|
| Lot No. | Rep 1 | Rep 2 | (CFU) |
| 102113RD-1 | + | + | 31 |
| 102113RD-2 | + | + | |

The above results confirm that the tested compositions both passed the AOAC Germicidal Spray Test for Trichophyton mentagrophytes with a contact time of 2 minutes at room temperature (20° C.), as there was no visible growth observed in 10 out of 10 of the subculture broth tubes per lot per microorganism and the controls met their stipulated criteria.

Accordingly, these test confirm that these representative embodiments of the instant invention demonstrate microbial efficacy against several common virulent microorganisms and have utility for the disinfection of surfaces and microbial colonies present thereon.

### Example Embodiments

Table 11 shows some additional embodiments of the instant invention, showing useful ranges of the various ingredients that can be formulated into a disinfecting and toxant treating composition suitable for use according to the current disclosure.

TABLE 11

| | Composition | | | | | | | | | | |
|---|---|---|---|---|---|---|---|---|---|---|---|
| Ingredient | 15 | 16 | 17 | 18 | 19 | 20 | 21 | 22 | 23 | 24 | 25 |
| Mayoquest 1500 (1) | 0.040 | 0.045 | 0.050 | 0.053 | 0.055 | | | | | | |
| Dequest 2010 (2) | | | | | | 0.040 | 0.045 | 0.045 | 0.050 | 0.055 | 0.048 |
| Peroxide (3) | 5.000 | 5.000 | 5.000 | 5.000 | 5.000 | 4.500 | 4.750 | 5.000 | 5.500 | 5.750 | 5.350 |
| Maquat 1412 (4) | 0.100 | 0.110 | 0.120 | 0.125 | 0.130 | | | | | | 0.117 |
| Maquat 2420 (5) | | | | | | 0.100 | 0.110 | 0.120 | 0.125 | 0.130 | |
| pH Adj (6) | | | | | | A | B | B | C | C | B |
| Water, Deionized | 94.860 | 94.845 | 94.830 | 94.822 | 94.815 | 95.360 | 95.095 | 94.835 | 94.325 | 94.065 | 94.485 |
| Ratio P:S (7) | 125.0 | 111.1 | 100.0 | 94.3 | 90.9 | 112.5 | 105.5 | 111.1 | 110.0 | 104.5 | 111.5 |
| pH (Final) | 2.2 | 2.2 | 2.2 | 2.2 | 2.2 | 2.1 | 2.2 | 2.3 | 2.4 | 2.5 | 2.2 |

(1) Mayoquest 1500, HEDP, 60%
(2) Dequest 2010, HEDP, 60%
(3) PeroxyChemB-50, Hydrogen Peroxide, 50%
(4) Maquat ® 1412-50% is a 50% active ADBAC
(5) Maquat ® 2420-50% is a 50% active ADBAC
(6) Identifier of material used to adjust pH: A = 75% phosphoric acid; B = 50% caustic soda; C = 50% KOH
(7) Ratio of Peroxide/Sequestrant (wt. %/wt. %, as 100% active)

### Methods of Use

In general, embodiments of the instant invention, formulated into single aqueous compositions, can be applied directly to an object or surface as is, without dilution, and allowed to wet and contact that object or surface for a proscribed time period, the contact time being between 10 seconds to about 10 minutes, or alternatively between 30 seconds and 5 minutes, or alternatively between 30 seconds and 2 minutes, or yet alternatively between 30 seconds and 1 minute, depending on the degree of microbial or toxant contamination present on the surface to be treated.

Embodiments of the instant invention can be applied as a fine spray, such as for example in the form of an aerosol or fine droplets delivered through a nozzle present on a pressurized can, conventional trigger sprayer or spray pump dispensing device, or alternatively applied as a liquid film using a wetted wipe, non-woven applicator, or other similar device. Inventive compositions can also be pre-applied immediately prior to use onto a foam, sponge, wipe, or non-woven matrix to form a saturated or partially saturated treatment means in which to transfer the inventive compositions to a surface and spread the materials uniformly across the surface to be treated. In addition, embodiments of the instant invention can be used in a bath treatment device, wherein objects to be treated can be submerged or coated with the inventive compositions for the desired length of time.

Surfaces that can be treated include any hard, non-porous surface, for example including, but not limited to glass, plastic, metal, stainless steel, limestone, granite, tile, concrete, marble, porcelain, coated surfaces, and combinations thereof. Suitable surfaces that can be treated with embodiments of the instant invention also include soft surfaces, for example including, but not limited to clothing, textiles, medical fabrics, bandages, drapes, sheets, bedding, pillows, and the like, provided that some retention of the material through wetting is acceptable under the circumstances.

In general, it is desirous for the contacted surfaces to remain wet or at least partially wetted during the proscribed treatment time, in order for the inventive compositions to contact the entirety of the surface and contact it for a time period sufficient to produce the desired effect.

General usage instructions for an aliquot of treatment composition corresponding to any one embodiment of the instant invention as disclosed herein are as follows:

1. Do not dilute.

2. Wet surface thoroughly using an application device such as a sprayer, sponge, mop, etc. Surface must remain wet for two minutes.

3. Reapply if it dries out or becomes diluted by any liquid during the exposure time.

### Facilities Usage

Examples of facilities in which embodiments of the instant invention can be used include, but are not limited to, healthcare institutions, such as hospitals, surgical centers, dialysis centers, outpatient clinics, doctor's offices, cancer centers, nursing homes, dentist's office and exercise/physical therapy facilities; public safety facilities, such as fire and police departments, holding cells, ambulance services, emergency medical services and ambulances, jails and prisons; educational institutions such as schools, gymnasiums and universities, and hospitality venues, such as airplanes, airports, train stations, resorts, hotels, motels, health clubs, subway stations, subway trains and cruise ships.

The above illustrations provide many different compositional embodiments or embodiments for implementing different features of the invention. Specific embodiments of components and processes are described to help clarify the invention. These are, of course, merely embodiments and are not intended to limit the invention from that described in the claims.

Although the invention is illustrated and described herein as embodied in one or more specific examples, it is nevertheless not intended to be limited to the details shown, since various modifications, compositional changes and process variations may be made therein without departing from the spirit of the invention and yet remaining within the scope and range of equivalents of the claims. Accordingly, it is appropriate that the appended claims be construed broadly and in a manner consistent with the scope of the invention, as set forth in the following claims.

I claim:

1. A formulation for use in neutralization of at least one microorganism or toxant present on a surface, said formulation comprising:
(a) a biocidal cationic surfactant;
(b) a source of hydrogen peroxide; wherein said source of hydrogen peroxide is selected from the group consisting of hydrogen peroxide, peracetic acid, sodium perborate, sodium peroxypyrophosphate, sodium peroxysilicate, and sodium percarbonate; and
(c) at least one oxidant-stable stabilizer;
wherein said stabilizer is present at a level sufficient to stabilize said formulation against significant loss of said hydrogen peroxide for up to one year storage under ambient room temperature conditions; and
wherein said stabilizer is present at or below a selected ratio with respect to said source of hydrogen peroxide enabling for accelerated oxidant activity when said formulation is applied to said surface at time of use; and
wherein said ratio of hydrogen peroxide to stabilizer is less than or equal to 125:1.

2. The formulation of claim 1, wherein said ratio of hydrogen peroxide to stabilizer is equal to or between 90:1 and 125:1.

3. The formulation of claim 1, wherein said ratio of hydrogen peroxide to stabilizer is between 100:1 and 125:1

4. The formulation of claim 1, wherein said biocidal cationic surfactant is selected from the group consisting of dialkyl dimethyl ammonium salts, dialkyl diethyl ammonium salts, alkyl dialkylbenzyl ammonium salts, alkyl pyridinium ammonium salts, polyhexamethylene biguanide hydrochloride salts, polyhexamethylene guanidine hydrochloride salts, dimethyldidecyl ammonium chloride salts, benzalkonium chloride, benzethonium chloride, chlorhexidine digluconate, poly (dimethyl butenyl ammonium chloride salts, alpha, omega-bis (triethanol-ammonium chloride salts, poly (oxyethylene) (dimethylimino) ethylene (dimethylimino) ethylene dichloride, dequalinium chloride, polyquaternium 2, hexetidine, cetyl pyridinium chloride, tetrakis (hydroxy methyl) phosphonium sulfate, ethanediyl-α, w-bis (dodecyldimethyl) ammonium halides, quaternary ammonium dendrimeric biocides, and combinations thereof; wherein said alkyl substituents have saturated carbon chain lengths of 1 to 18, and combinations thereof.

5. The formulation of claim 4, wherein said biocidal cationic surfactant is selected from an N-alkyl dimethylbenzyl ammonium salt; wherein said N-alkyl substituent is a saturated alkyl substituent having a chain length equal to or between 12 and 18 alkane units; and wherein said salt is a counterion or adduct selected from a halide, hydroxide, sulfate, bicarbonate, carbonate or hydrogen chloride; and wherein said halide is either a chloride or bromide counterion.

6. The formulation of claim 5, wherein said biocidal cationic surfactant is selected from the group consisting of mixed alkyl substituent chain lengths, wherein said mixed alkyl substituent chain lengths include dodecyl, tetradecyl, and hexadecyl substituents.

7. The formulation of claim 6, wherein said N-alkyl dimethylbenzyl ammonium salt has a halide counterion; and wherein said N-alkyl substituent is a mixture of 40% dodecyl (C12), 50% tetradecyl (C14) and 10% hexadecyl (C16) substituents; and wherein said halide is either a chloride or bromide counterion.

8. The formulation of claim 4, wherein said biocidal cationic surfactant is present at a level equal to or between 0.05 weight % and 0.20 weight %.

9. The formulation of claim 1, wherein said oxidant-stable stabilizer is selected from the group consisting of organic phosphonates, alkanehydroxyphosphonates, carboxylates, and mixtures thereof.

10. The formulation of claim 9, wherein said oxidant-stable stabilizer is selected from 1-hydroxethane-1,1-diphosphonic acid, amino-trismethylenephosphonic acid, diethylenetriamine penta(methylene phosphonic acid), diethylenetriamine penta(methylene phosphonic acid, hepta-sodium diethylenetriamine penta(methylene phosphonate, ethylenediaminetetraacetic acid, N-hydroxyethylene-diaminetetraacetic acid, nitrilotriacetic acid, N-hydroxyethyl-aminodiacetic acid, diethylene-triaminepentaacetic acid, methylglycinediacetic acid, alanine-N,N-diacetic acid, salts thereof, and combinations thereof.

11. The formulation of claim 10, wherein said oxidant-stable stabilizer is 1-hydroxethane-1,1-diphosphonic acid and salts thereof.

12. The formulation of claim 11, wherein said oxidant-stable stabilizer is selected from a 1-hydroxethane-1,1-diphosphonic acid and salts thereof; wherein said biocidal cationic surfactant is an N-alkyl dimethylbenzyl ammonium salt having a halide counterion; wherein said N-alkyl substituent is a mixture of 40% dodecyl (C12), 50% tetradecyl (C14) and 10% hexadecyl (C16) substituents; and wherein said halide is either a chloride or bromide counterion.

13. The formulation of claim 12, wherein the source of hydrogen peroxide is an aqueous hydrogen peroxide.

14. The formulation of claim 13, wherein the ratio of hydrogen peroxide to said oxidant-stable stabilizer is equal to or between 95:1 and 125:1.

15. A formulation for use in neutralization of at least one microorganism or toxant present on a surface, said formulation consisting of:
(a) a biocidal cationic surfactant being an N-alkyl dimethylbenzyl ammonium salt having a halide counterion; wherein said N-alkyl substituent is a mixture of 40% dodecyl (C12), 50% tetradecyl (C14) and 10% hexadecyl (C16) substituents; wherein said halide is either a chloride or bromide counterion;
(b) a source of hydrogen peroxide, wherein said source of hydrogen peroxide is selected from the group consisting of hydrogen peroxide, peracetic acid, sodium perborate, sodium peroxypyrophosphate, sodium peroxysilicate, and sodium percarbonate; and
(c) at least one oxidant-stable stabilizer being 1-hydroxethane-1,1-diphosphonic acid and salts thereof; wherein the ratio of said hydrogen peroxide to said stabilizer is equal to or between 95:1 and 125:1.

16. A formulation for use in neutralization of at least one microorganism or toxant present on a surface, said formulation consisting essentially of:
(a) N-alkyl dimethylbenzyl ammonium chloride wherein said N-alkyl substituent is a mixture of 40% dodecyl (C12), 50% tetradecyl (C14) and 10% hexadecyl (C16) present at a level equal to or between 0.10 weight % and 0.13 weight %;
(b) hydrogen peroxide present at a level equal to or between 4.5 weight % and 5.75 weight %; and
(c) an oxidant-stable stabilizer selected from 1-hydroxethane-1,1-diphosphonic acid and salts thereof;
wherein the ratio of said hydrogen peroxide to said oxidant-stable stabilizer is equal to or between 90:1 and 125:1.

17. A method of treating a surface to neutralize at least one bacterial or chemical toxant, comprising:
(a) applying a formulation to said surface in the form of a thin aqueous film; wherein said formulation comprises:
(i) a biocidal cationic surfactant;
(ii) a source of hydrogen peroxide; wherein said source of hydrogen peroxide is selected from the group consisting of hydrogen peroxide, peracetic acid, sodium perborate, sodium peroxypyrophosphate, sodium peroxysilicate, and sodium percarbonate;
(iii) at least one oxidant-stable stabilizer; wherein the ratio of said hydrogen peroxide to said oxidant-stable stabilizer is equal to or between 90:1 and 125:1;
(b) allowing said thin aqueous film to remain in contact with said surface for a proscribed period of time of between 30 seconds and 10 minutes as necessary to neutralize said bacterial or chemical toxant; and
(c) either removing said thin aqueous film from said surface or allowing said thin aqueous film to evaporate to dryness.

18. The method of claim 17, wherein said biocidal cationic surfactant is selected from an N-alkyl dimethylbenzyl ammonium salt; wherein said N-alkyl substituent is a saturated alkyl substituent having a chain length equal to or between 12 and 18 alkane units; and wherein said salt is a counterion or adduct selected from a halide, hydroxide, sulfate, bicarbonate, carbonate or hydrogen chloride; wherein said halide is either a chloride or bromide counterion; and wherein said oxidant-stable stabilizer is selected from 1-hydroxethane-1,1-diphosphonic acid, amino-trismethylenephosphonic acid, diethylenetriamine penta(methylene phosphonic acid), diethylenetriamine penta(methylene phosphonic acid, hepta-sodium diethylenetriamine penta(methylene phosphonate, ethylenediaminetetraacetic acid, N-hydroxyethylene-diaminetetraacetic acid, nitrilotriacetic acid, N-hydroxyethyl-aminodiacetic acid, diethylene-triaminepentaacetic acid, methylglycinediacetic acid, alanine-N,N-diacetic acid, salts thereof, and combinations thereof.

19. The method of claim 17, wherein said formulation comprises
(a) N-alkyl dimethylbenzyl ammonium chloride wherein said N-alkyl substituent is a mixture of 40% dodecyl (C12), 50% tetradecyl (C14) and 10% hexadecyl (C16) present at a level equal to or between 0.10 weight % and 0.13 weight %;
(b) hydrogen peroxide present at a level equal to or between 4.5 weight % and 5.75 weight %; and
(c) an oxidant-stable stabilizer selected from 1-hydroxethane-1,1-diphosphonic acid and salts thereof;
wherein the ratio of said hydrogen peroxide to said oxidant-stable stabilizer is equal to or between 90:1 and 125:1.

20. The method of claim 19, further comprising instructions for use of said formulation comprising directions to leave said formulation in contact with said surface for at least two minutes in order to effect a 4-log reduction with respect to a colony formation unit of at least one microorganism selected from the genus Escherichia coli, Staphylococcus aureus, Pseudomonas aeruginosa, Salmonella enterica, and subgenera thereof, and subspecies thereof.
